(19) United States
(12) Patent Application Publication
(10) Pub. No.: US 2023/0384332 A1
(43) Pub. Date: Nov. 30, 2023

(54) RAPID ASSAY FOR APOL1 G0 PROTEIN

(71) Applicant: AFFINERGY, LLC, Research Triangle Park, NC (US)

(72) Inventors: Martyn Kerry DARBY, Chapel Hill, NC (US); Patrick LESLIE, Morrisville, NC (US); Emily Vaughan, Indianapolis, IN (US); Isaac Gilliam SANFORD, Durham, NC (US); Matthew AXTMAN, Cary, NC (US)

(21) Appl. No.: 18/364,663

(22) Filed: Aug. 3, 2023

Related U.S. Application Data
(63) Continuation of application No. PCT/US22/15215, filed on Feb. 4, 2022.
(60) Provisional application No. 63/146,443, filed on Feb. 5, 2021.

(51) Int. Cl. G01N 33/92; G01N 33/543

(52) U.S. Cl. CPC G01N 33/92; G01N 33/54388; G01N 2333/775

(57) ABSTRACT

ELISA-type assays, lateral flow test strips, methods, and systems are provided for detecting APOL1 G0 in bodily samples including blood, serum, or plasma to provide a rapid rule-in test for the ApoL1 G0 genotype. The assays exploit the differential affinity of serum resistance-associated protein (SRA) for wild-type APOL1 (designated ‘G0’) over the other variants G1 and G2 to specifically detect APOL1 G0. Results with human plasma samples (N=130) from all six genotypes (G0/G0, G0/G1, G0/G2, G1/G2, G2/G2, G1/G1) show the assay can detect APOL1 G0 from plasma with 100% concordance with genotyping. The assay fulfills an unmet need for a rapid test (i.e., within about an hour) for determining APOL1 variant status in deceased kidney donors which represent 72% of donated kidneys.

### CROSS-REFERENCE TO RELATED APPLICATIONS

This application is a continuation application of International Application No. PCT/US2022/015215, filed Feb. 4, 2022, which claims priority to U.S. Provisional Patent Application No. 63/146,443, filed on Feb. 5, 2021, the entire contents of which are all incorporated by reference herein in their entirety.

### STATEMENT OF GOVERNMENTAL INTEREST

This invention was made with Government support under grant 1R43MD013402 awarded by the National Institutes of Health. The Government has certain rights in the invention.

### TECHNICAL FIELD

The present disclosure relates to assays, lateral flow test strips, and rapid methods for detection of ApoL1 G0 in biological samples such as, for example, blood, serum, or plasma samples for rapid rule-in tests, including tests for screening potential kidney donors.

### BACKGROUND

African Americans are disproportionately affected by chronic kidney disease (CKD) and end stage renal disease (ESRD)^{[1]}; while 35% of patients on dialysis are African American, only 13.2% of the U.S. population is African American^{[2]}. One factor contributing to this disparity is genetic variation in apolipoprotein L1 (APOL1), a circulating apolipoprotein originally identified through its role in resistance to African sleeping sickness caused by Trypanosoma brucei rhodesiense infection^{[4, 5]}.

There are three well-characterized APOL1 allelic variants: G0 (wild-type), G1 and G2E^{[4]}. The APOL1 G1 and G2 variants confer resistance to infection through reduced interaction with the serum-resistance associated (SRA) protein found in T. brucei rhodesiense^{[4, 6, 17]}. Although the G1 and G2 APOL1 variants are protective against African sleeping sickness, they are also associated with 5-29 times higher odds for the development of severe kidney disease including focal segmental glomerulosclerosis, CKD and ESRD^{[8-13]}.

In the transplant setting, the presence of any two risk alleles (G1/G1, G2/G2, or G1/G2) in a donor kidney is also associated with a two times greater risk of graft failure, while the presence of even one G0 allele eliminates this risk, regardless of recipient APOL1 status^{[14-16, 18]}. Further, living donors with two APOL1 risk alleles are at increased risk of accelerated impairment of renal function and ESRD after donating a kidney^{[19]}. However, no existing APOL1 test is compatible with the timeframe required for the evaluation of deceased kidney donors (72% of donated kidneys), while available gene sequencing and mass spectrometry (MS)-based tests for living donors are complex, time-consuming, and costly.

According to current guidelines on ‘Effective Practices in Broader Distribution’ from the Organ Procurement and Transportation Network (OPTN) of the US Department of Health and Human Services, transplant centers have just one hour to evaluate deceased organ donor offers (including kidney offers) and enter a provisional acceptance or refusal^{[20]}. This guidance is part of a policy that is intended to increase efficacy of organ allocation, donor and recipient matching, transportation logistics over a broader distribution range, and organ recovery. However, none of the currently available tests are compatible with these guidelines for evaluating deceased donors prior to transplant.

In 2018, 74% of US nephrologists and transplant surgeons of the American Society of Nephrology, the American Society of Transplantation, and the American Society of Transplant Surgeons surveyed indicated that APOL1 testing would offer better clinical information about a kidney donor's eligibility than existing evaluation approaches, such as the Kidney Donor Risk Index (KDRI)^{[21]}. Importantly, the KDRI was implemented in 2014, before the APOL1 effect in kidney donor transplantation was identified. A 2017 study calculated that as many as 10,000 donated kidneys discarded over 5 years could have been transplanted if APOL1 variant status had been used in place of African American race as a risk factor on the KDRI^{[3]}. As of 2016, 18% of donated kidneys are still being discarded, despite the fact that the number of patients waiting for a kidney (95,456) far exceeds the number of transplants (20,161) annually^{[2]}.

The current gold standard for determining APOL1 variant status for donor risk stratification is through targeted gene sequencing of exon 6^{[22]}, although liquid chromatography-mass spectrometry (LC-MS/MS)-based tests can also be used to determine APOL1 protein phenotype in circulation (LabCorp). Both of these methods have long turnaround times (1-3 weeks depending on the lab). While a faster, polymerase chain reaction (PCR) gene sequencing test has been developed, this lab-based test is currently only available to clinicians in a limited number of US states, has a turnaround time of 1 day (excluding shipping), and like targeted sequencing requires quality genomic DNA, which can impact accuracy and reproducibility. None of the available methods for APOL1 testing are compatible with the pre-transplant assessment of deceased kidney donors, >70% of all kidney donors^{[2]}, as they cannot provide results within 1 hour of organ offer notification^{[20]}. Additionally, protocols to assess APOL1 status by MS or gene sequencing are complicated, multi-step processes and transplant labs may not have the experience or expertise to perform and analyze these tests with the rigor required^{[23]}. The validation and quality control for APOL1 genotyping is more stringent than is typically required of these labs which routinely perform human leukocyte antigen (HLA) testing for donor matching^{[23]}.

Thus, there remains an unmet need for a method of rapidly measuring APOL1 variant status such as, for example, in samples from living and deceased kidney donors. The present disclosure provides such a method.

### SUMMARY

In one aspect the present disclosure provides a method for detecting wild-type ApoL1 protein (G0). The method includes incubating a sample from a subject being assessed for an ApoL1 G0 protein with: i) a first binding partner comprising a serum resistance-associated (SRA) protein that selectively binds APOL1 G0 over G1 and G2, and ii) a second binding partner comprising an ApoL1 specific binding partner. In the method, one of either the first or the second binding partner is immobilized on a solid phase, and the first or the second specific binding partner that is not immobilized on the solid phase has a detectable label. The ApoL1 G0 and the first and second binding partners form a solid phase binding complex during the incubation. The assay includes separating the solid phase binding complex from the unbound first or second binding partner comprising the detectable label. The detectable label associated with the separated complex indicates the presence of ApoL1 G0 in the sample. In some examples, a predetermined threshold for detecting the presence of ApoL1 G0 is about 7 nM in the sample. The incubating can, in some cases, range from a period of about 1 minute to about 1 hour.

In one embodiment of the present disclosure, a system is provided for detecting wild-type ApoL1 protein (G0), the system including: (a) an incubation vessel; (b) a reagent dispensing module; and (c) software to execute the method for detecting wild-type ApoL1 protein (G0) described above, wherein the method is executed robotically.

In the method and system, the sample can include a bodily fluid, such as, for example, blood, plasma, or serum, and combinations thereof. In certain instances, the bodily fluid can be at a dilution of 3-20%.

In one embodiment of the method and system, the solid phase includes microparticles, nanocellulose beads, or a surface of the incubating well or chamber. In one example, the microparticles are adsorbed with mouse anti-HA antibody and then recombinant SRA-Fc-HA.

In some examples of the method and system, the second ApoL1 specific binding partner includes an anti-ApoL1 antibody or a fragment or derivative thereof, an anti-mouse IgG antibody, a phage, or a peptide.

In some instances of the method and system, the detectable label includes an enzyme, oligonucleotide, nanoparticle, visible dye or colored compound, chemiluminophore, fluorophore, fluorescence quencher, chemiluminescence quencher, or biotin, and combinations thereof.

In one embodiment of the method and system, the subject is a potential kidney donor.

In one aspect the disclosure provides a lateral flow test strip (LFTS) for detecting wild-type APOL1 protein (G0). The LFTS includes: (a) a sample receiving pad for receiving a liquid sample; (b) a conjugate test pad in liquid communication with the sample receiving pad and downstream in flow direction from the sample receiving pad, wherein the conjugate test pad comprises a capture reagent deposited thereon, and wherein the capture reagent comprises (i) a serum resistance-associated protein (SRA) that selectively binds APOL1 G0 over G1 and G2, and (ii) a detectable reporting group; and (c) a nitrocellulose membrane in liquid communication with the conjugate pad and downstream in flow direction from the conjugate pad, wherein the nitrocellulose membrane includes at a first position a first detection reagent immobilized thereon, wherein the first detection reagent includes an APOL1 specific binding partner. In another embodiment of the present disclosure, a LFTS is provided for detecting wild-type APOL1 protein (G0) that includes: (a) a sample receiving pad for receiving a liquid sample; (b) a conjugate test pad in liquid communication with the sample receiving pad and downstream in flow direction from the sample receiving pad, wherein the conjugate test pad comprises a capture reagent deposited thereon, and wherein the capture reagent comprises (i) an APOL1 specific binding partner, and (ii) a detectable reporting group; and (c) a nitrocellulose membrane in liquid communication with the conjugate pad and downstream in flow direction from the conjugate pad, wherein the nitrocellulose membrane includes at a first position a first detection reagent immobilized thereon, wherein the first detection reagent includes a serum resistance-associated protein (SRA) that selectively binds APOL1 G0 over G1 and G2.

In one aspect, the present disclosure provides a system for detecting wild-type APOL1 protein (G0). The system includes: (a) either embodiment of the LFTS described above; and (b) a test reader to quantitatively determine if the amount of G0 present at the first position is above a predetermined threshold.

In some instances of the LFTS and the system, the liquid sample includes a bodily fluid. The bodily fluid can include, for example, blood, serum, or plasma, and combinations thereof.

In various embodiments of the LFTS and the system, the nitrocellulose membrane further includes a second detection reagent immobilized thereon at a second position downstream from the first detection reagent. In these embodiments, the second detection reagent is an antibody or a fragment or derivative thereof, a phage or a peptide that binds the capture reagent irrespective of whether the capture reagent is bound to APOL1 G0. In certain instances, the second detection reagent includes anti-mouse IgG antibody.

In some examples of the LFTS and the system, the APOL1 specific binding partner comprises an anti-ApoL1 antibody or a fragment or derivative thereof, an anti-mouse IgG antibody, a phage, or a peptide.

In one embodiment of the LFTS and the system, the capture reagent deposited on the conjugate test pad includes microparticles with the SRA or the APOL1 specific binding partner adsorbed or conjugated thereto. The microparticles can be adsorbed with mouse anti-HA antibody and then recombinant SRA-Fc-HA. In some instances, the microparticles include nanocellulose beads.

In one example of the LFTS and the system, the detectable reporting group is a visible dye or colored compound.

In one aspect the disclosure provides a method of assessing APOL1 status in a subject. The method includes (a) depositing a liquid sample from a subject onto the sample loading pad of any of the lateral flow test strips provided herein; and (b) determining that the subject has at least one wild-type APOL1 G0 allele when the detectable reporting group is visible at the first position, resulting in a positive test result.

### BRIEF DESCRIPTION OF THE FIGURES

FIGS. 1A-1B are schematic diagrams illustrating how recombinant serum resistance-associated protein (SRA) serves as a specific capture reagent for the APOL1 G0 in patient plasma. FIG. 1A shows the assay format. FIG. 1B shows the lateral flow test assay format.

FIG. 2 is a schematic showing recombinant proteins according to one or more embodiments of the present disclosure.

FIG. 3 is a Western blot of recombinant APOL1 variants incubated with immobilized SRA-Fc-HA on Protein G beads showing that recombinant SRA selectively binds APOL1 G0 over G1 and G2 providing the required specificity for the assay. Open triangle indicates the correct molecular weight of full length APOL1. Molecular weight markers in kilodaltons.

FIGS. 4A-4B are graphs showing an ELISA type assay of SRA-FLAG immobilized to anti-FLAG antibody after incubation with increasing amounts of recombinant APOL1 proteins according to one or more embodiments of the present disclosure. FIG. 4A is a dose response curve with APOL1 G0. FIG. 4B is a comparison of the dose-response with normalized amounts of APOL1 G0, G1 and G2, showing little response from the G1 and G2 lysates.

FIG. 5 is a graph showing the results of an SRA-based ELISA assay that correctly identified the presence or absence of APOL1 G0 in 98% of patient samples tested according to one or more embodiments of the present disclosure.

FIG. 6 is a graph showing the results of an SRA-based ELISA assay that correctly identified the presence or absence of APOL1 G0 in 100% of patient samples tested according to one or more embodiments of the present disclosure.

FIG. 7 is a photographic image showing results of conversion of the SRA-based ELISA assay to a lateral flow format using nitrocellulose membranes with anti-ApoL1 (“test line”) and anti-mouse IgG (“control line”) according to one or more embodiments of the present disclosure.

FIG. 8 is a drawing illustrating a lateral flow test assay for APOL1 G0 according to one or more embodiments of the present disclosure.

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the descriptions provided herein. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary, and so forth.

Throughout this specification and the claims, the terms “comprise,” “comprises,” and “comprising” are used in a non-exclusive sense, except where the context requires otherwise. Likewise, the terms “having” and “including” and their grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range. In addition, as used herein, the term “about”, when referring to a value can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed compositions and methods. Where particular values are described in the application and claims, unless otherwise stated the term “about” meaning within an acceptable error range for the particular value should be assumed.

Throughout this specification and the claims, the term “subject” includes humans and animals and can be used interchangeably with the term “human” and the term “patient”.

The terms “assay” and “method” are herein used interchangeably for the purposes of the specification and claims.

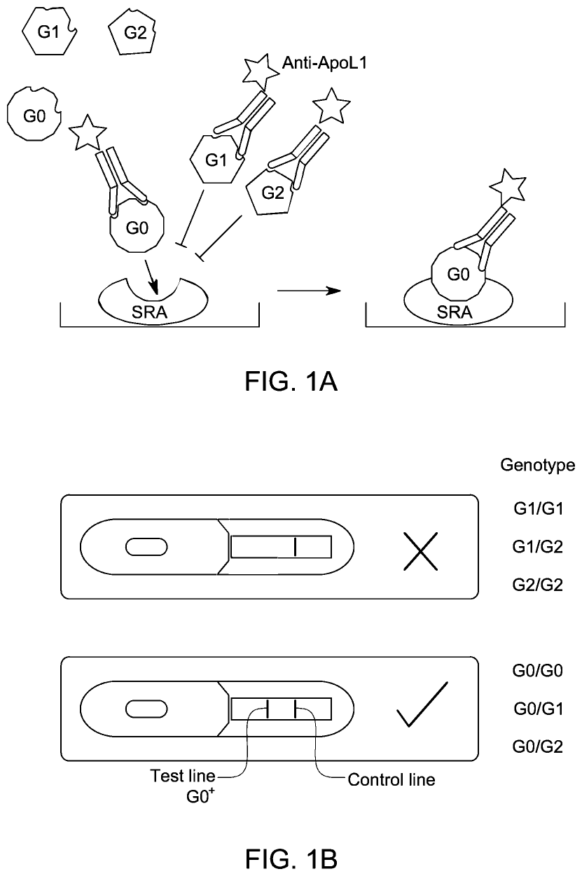

The present disclosure provides, in one aspect, a sandwich ELISA type assay (FIG. 1A) that exploits the differential affinity of serum resistance-associated protein (SRA) for the wild-type variant of APOL1 (designated ‘G0’) over the other variants G1 and G2^{[4, 6]} to specifically detect APOL1 G0 from plasma. In some embodiments, the assay can specifically detect APOL1 G0 from plasma with at least 98% concordance with genotyping. In the assay shown in FIG. 1A, recombinant SRA serves as a specific capture reagent for the APOL1 G0 variant in plasma, including patient plasma.

As illustrated in the schematic in FIG. 1B, in another aspect the assay is provided as a point of care test lateral flow assay or lateral flow test strip (LFTS) requiring only a drop of blood to enable the detection of at least one G0 allele. In this example of the assay, the SRA beads bind well only to ApoL1 G0 and the beads are captured on an anti-ApoL1 “test line”. The term “test line” is used interchangeably herein for the purposes of the specification and claims, as “a first position comprising a first detection reagent”. Successful bead migration is, in this example, recorded by an anti-mouse IgG “control line”. The term “control line” is used interchangeably herein for the purposes of the specification and claims, as “a second position comprising a second detection reagent”.

A significant advantage of the assay and the lateral flow test provided herein, is that results from these tests will be available within about 30 minutes of blood collection from a subject, making it the only test compatible with current guidelines for evaluating deceased kidney donors prior to transplant.

Structural alterations within the C-terminal coiled coil domain of APOL1 G1 and G2 destabilize the two-helix bundle of this domain^{[24]}, which may contribute to the lower affinity of SRA for these variants in comparison with wild-type G0^{[4, 7]}, APOL1 G1 has two amino acid substitutions (S342G/I384M) and G2 has a two-amino acid deletion (ΔN388Y389)^{[4]}. One study examining peptides corresponding to the C-terminal coiled coil domains of G1, G2, and G0 found a maximum K_{D} of ˜4 nM at pH 4.5 (lysosomal pH) for SRA binding the G0 peptide with two- and five-fold reductions in binding to the G1 and G2 peptides, respectively^{[7]}.

The development of a clinically useful immunoassay for the rapid detection of APOL1 G0 for kidney donor risk stratification has been hindered by the relatively subtle differences in the amino acid alterations among variants. Additionally, the non-invasive, blood-based detection of APOL1 variant status requires reagents capable of detecting APOL1 within two distinct native multiprotein complexes in circulation (a 500 kDa lipid-rich complex and 1000 kDa lipid-poor complex), which presumably limits the number of available epitopes in vivo.

The assays, methods, lateral flow test strips, and systems provided herein make use of the SRA protein from Trypanosoma brucei as a capture reagent for human APOL1 G0. Given that a single copy of the APOL1 G0 allele is sufficient to eliminate all risk associated with the G1 or G2 alleles^{[14-16, 18]}, the present inventors recognized that it is not necessary to perform a complete genotypic analysis for accurate risk stratification in kidney donors; it is sufficient to determine the presence or absence of G0. Use of recombinant SRA in the presently disclosed methods and systems allows for the rapid and selective detection of APOL1 G0 over the G1 and G2 variants in patient plasma and other biological samples. Such selectivity has failed to be achieved by antibodies thus far, blocking the development of traditional immunoassays. In a lateral flow format as illustrated in FIG. 1B, the presently disclosed method can yield results in less than 30 min. Another advantage of the methods, LFTS's, and systems provided herein is that measuring the APOL1 G0 protein in blood (or measuring the APOL1 G0 protein in another type of bodily sample) avoids any possible discordance between genotype and protein phenotype resulting from inhibition of expression of one allele.

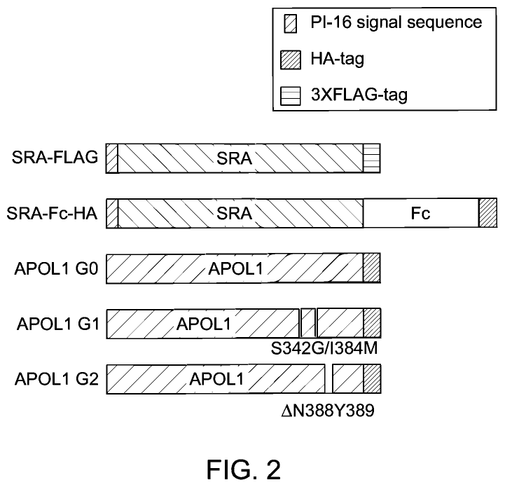

In one embodiment of the present disclosure, an expression construct for the T. brucei rhodesiense SRA protein is provided where the putative N-terminal secretion sequence is replaced with the N-terminal secretion sequence of PI-16 and a C-terminal 3× FLAG sequence or Fc-HA sequence is added to the C-terminus as described in Example 1. The sequence of the final construct is illustrated in FIG. 2. However, the SRA protein of the present disclosure is not limited to a particular sequence or recombinant construct. The SRA protein of the present disclosure can be wildtype SRA, or a variant or fragment thereof, that retains the function of the wildtype protein. More specifically, the retained function of the SRA protein is binding to APOL1 G0 with a selectivity of at least about 10-fold over binding to G1 and G2.

In other embodiments of the present disclosure, full length APOL1 can be constructed similarly to SRA with appropriate amino acid changes or deletions to create the G1 and G2 variants and these constructs are also shown in FIG. 2.

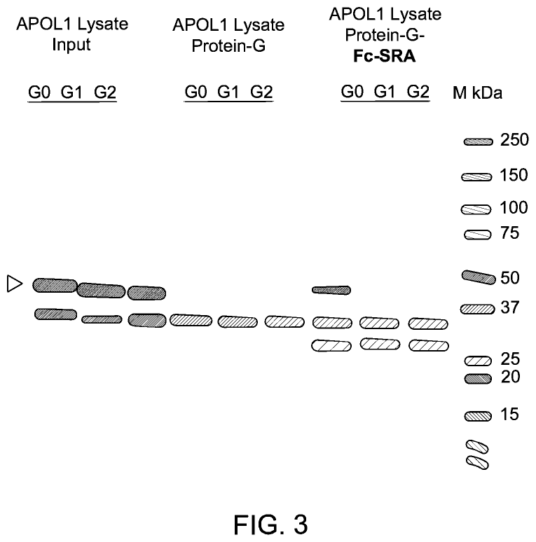

In one example, APOL1 pull-down experiments, performed with immobilized SRA-Fc-HA similar to an immunoprecipitation, demonstrate the binding specificity of the SRA capture protein for APOL1 G0 (see Example 2). Lysates prepared from cells expressing APOL1 variants were normalized with respect to APOL1 concentration and incubated with Protein G Sepharose previously bound with or without SRA-Fc-HA. The data in FIG. 3 show that SRA binds APOL1 G0 but not the G1 or G2 protein variants thus providing the required specificity for the assay.

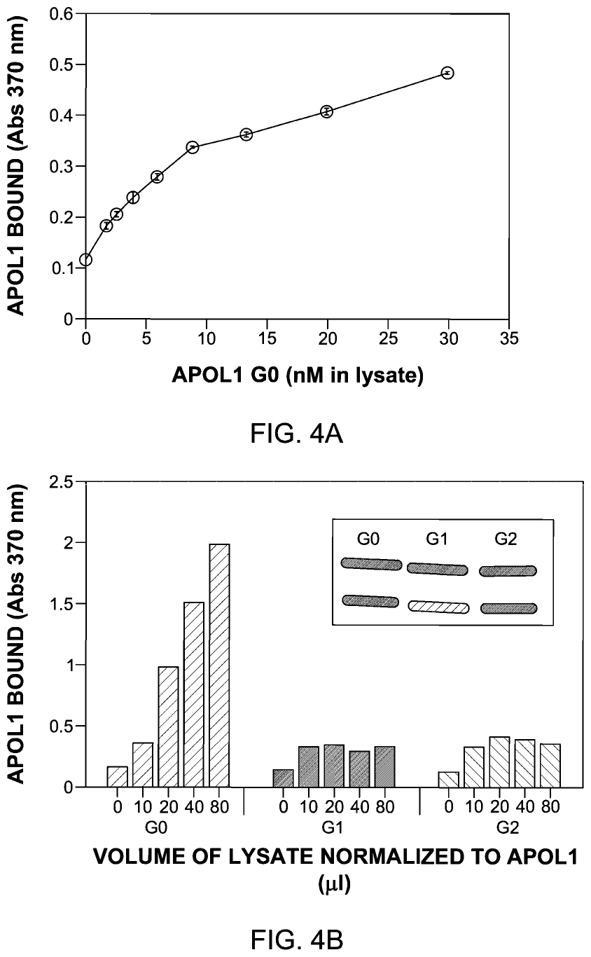

The specificity and sensitivity of the SRA-based assays, methods, lateral flow test strips, and systems provided herein is described in Example 3. FIG. 4A is a graph of a dose response curve for recombinant APOL1 G0 binding to SRA in one embodiment of the present disclosure. FIG. 4B is a graph showing that the SRA-based assay is specific for the G0 variant of APOL1. Specifically, immobilized SRA-FLAG incubated with increasing and equivalent amounts of G0, G1, and G2 isotype extracts, normalized by comparing Western blot intensities shows little response from the G1 and G2 lysates. These data illustrate the specificity of SRA for the G0 variant.

In some embodiments, a colorimetric substrate is utilized as a detection method in the SRA-based assays, methods, lateral flow test strips, and systems of the present disclosure. In one example, anti-FLAG antibody is adsorbed to a microtiter plate to present SRA-FLAG capture reagent. An anti-APOL1 antibody in combination with HRP conjugated anti-rabbit IgG is used to detect APOL1 bound to the SRA. Plasma diluted to 5% is incubated with immobilized SRA-FLAG. APOL1 binding is detected by anti-APOL1 and anti-rabbit HRP and TMB colorimetric substrate.

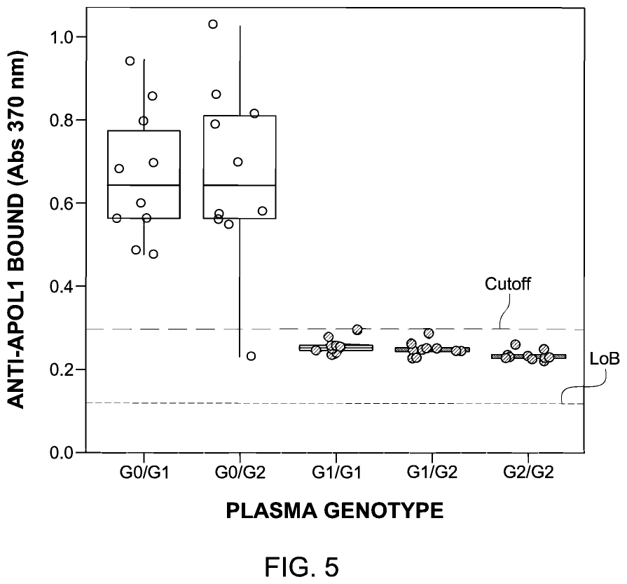

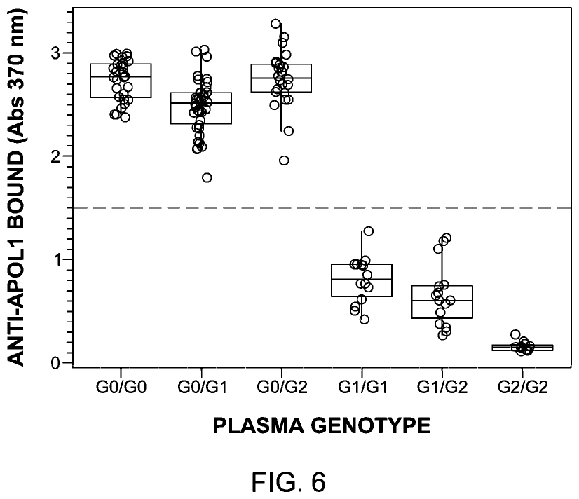

Example 3 describes validation of the assay performance in one embodiment of the present disclosure in clinically obtained plasma samples of all potential APOL1 risk allele genotype combinations: G0/G0, G0/G1, G0/G2, G1/G2, G2/G2, and G1/G1. Two separate experiments are described in Example 3 and the results are shown in FIG. 5 and FIG. 6. The results in FIG. 5 and FIG. 6 show that the SRA-based ELISA assay can correctly identify the presence or absence of APOL1 G0 in 100% of patient samples tested. For example, in the FIG. 6 assay, plasma was utilized from N=130 African Americans representing all potential genotypes bearing 0-2 risk alleles: APOL1 G0/G0, G0/G1, G0/G2 (non-risk states) and APOL1 G1/G1, G1/G2, G2/G2 (risk states). These plasma samples had been previously genotyped. The assay was tested using a 10% dilution of each plasma sample in sodium acetate buffer. The results in FIG. 6 show that each of the six genotypes is clearly defined by the scale of response in the assay. The concordance of the assay results illustrated in FIG. 6 with APOL1 genotype determined by sequencing is 100% with 90/90 individuals correctly identified as non-risk (signal greater than 1.5, positive for G0) and 40/40 identified as risk (signal less than 1.5, negative for G0). APOL1 G0 concentrations within patient samples can range from 100-1800 nM.^{[25]} In this embodiment of the SRA-based assay, the signal is greater than 3-fold above noise at approximately 7 nM, indicating that the assay performs well below the range required for accurate detection of APOL1 G0 in the clinic. The LoB (qualitative cutoff) of the assay is demonstrated in clinical samples, as shown in FIG. 5 and FIG. 6. In some embodiments, the SRA-based assay of the present disclosure can correctly identify the presence or absence of APOL1 G0 in 100% of patient samples tested.

The use of plasma in the assays, methods, and systems of the present disclosure is not limited to a dilution to 5%. In one example, plasma shows a positive response at 3-20% dilution with a maximum response around 5% dilution. Ammonium sulfate fractionation of plasma suggests the presence of an inhibitor can be the cause for reduction in signal above 20% plasma. The sensitivity of the assay circumvents this problem and sample dilution further decreases the risk of matrix interference. The assay can also be fully functional in diluted or undiluted serum, in 10% whole blood, or in undiluted whole blood.

In one aspect, the present disclosure provides an assay for detecting wild-type ApoL1 protein (G0). The assay includes (a) incubating a sample from a subject being assessed for an ApoL1 G0 protein with: i) a first binding partner comprising a serum resistance-associated (SRA) protein that selectively binds APOL1 G0 over G1 and G2, and ii) a second binding partner comprising an ApoL1 specific binding partner. In the assay, one of either the first or the second binding partner is immobilized on a solid phase, and the first or the second specific binding partner that is not immobilized on the solid phase has a detectable label. The ApoL1 G0 and the first and second binding partners form a solid phase binding complex during the incubation. The assay includes (b) separating the solid phase binding complex from the unbound first or second binding partner comprising the detectable label. The detectable label associated with the separated complex indicates the presence of ApoL1 G0 in the sample.

In another embodiment of the present disclosure, a system is provided for detecting wild-type ApoL1 protein (G0), the system including: (a) an incubation vessel; (b) a reagent dispensing module; and (c) software to execute the method for detecting wild-type ApoL1 protein (G0) described above, wherein the method is executed robotically.

In the assay, the incubating can be for a period of 1 minute to 1 hour.

In the assay, the subject can be a potential kidney donor, including a potential deceased kidney donor. The sample can be a bodily fluid from the subject including, for example, a blood, serum, or plasma sample. The bodily fluid can be used in the assay diluted or undiluted, including use at a dilution ranging from 3-20%.

As described above, the SRA protein of the present disclosure is not limited to a particular sequence or recombinant construct, so long as the SRA protein retains the function of binding to APOL1 G0 with a selectivity of at least about 10-fold over binding to G1 and G2.

In the assay, the second ApoL1 specific binding partner can include any suitable ApoL1 specific binding partner including, but not limited to, an anti-ApoL1 antibody or a fragment or derivative thereof, or a phage or peptide.

In the assay, the solid phase can include, but is not limited to, microparticles, nanocellulose beads, or a surface of a well or chamber in which the incubating is being performed. The SRA protein and the ApoL1 specific binding partner can be immobilized on the solid phase in any suitable manner as known to those of skill in the field.

In the assay, the detectable label can include any suitable label including, but not limited to, an enzyme, oligonucleotide, nanoparticle, visible dye or colored compound, chemiluminophore, fluorophore, fluorescence quencher, chemiluminescence quencher, or biotin.

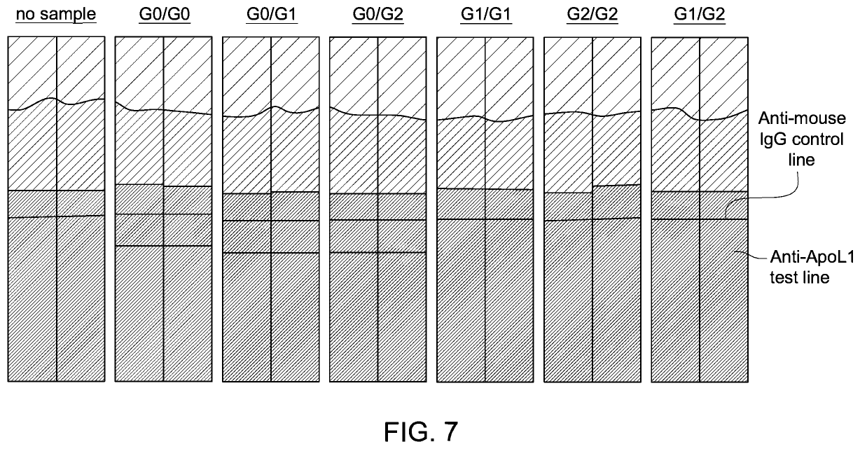

In another embodiment of the present disclosure, the SRA-based assay is provided in a lateral flow format. Example 4 and FIG. 7 illustrate that the assay in lateral flow format can provide a rapid rule-in test for the ApoL1 G0 genotype. Example 4 describes one example of the SRA-based assay in a lateral flow format, using nitrocellulose membranes prepared with anti-ApoL1 on the test line and anti-mouse IgG (which binds to anti-FLAG antibody) on the control line. Other embodiments of the assay in lateral flow format include using nitrocellulose membranes prepared with SRA that selectively binds APOL1 G0 over G1 and G2, rather than anti-ApoL1, on the test line. In one example of the assay in lateral flow format, plasma samples from patients of all six possible APOL1 genotypes are diluted to 10% with PBST and mixed with nanocellulose beads adsorbed with first mouse anti-HA antibody and then recombinant SRA-Fc-HA (as illustrated in FIG. 4). Nitrocellulose strips with wicks are placed upright in the bead/plasma suspension and beads allowed to migrate up the strip. Beads are retained by the ApoL1 test spot only if the plasma is a genotype containing G0 (i.e., G0/G0, G0/G1, or G0/G2). Beads mixed with G1 or G2 genotype plasma (i.e., G1/G2, G2/G2, or G1/G1) do not bind anti-ApoL1, since SRA binds more weakly to these variants, but do bind to the positive control line showing that the SRA-beads migrated past the test spot (see FIG. 7). The results in FIG. 7 show that the lateral flow format of the assay retains selectivity of SRA for the G0 isoform of ApoL1. Duplicate samples of each plasma were mixed with SRA-Fc-HA beads and allowed to migrate across the test area. The arrow indicates the direction of bead flow.

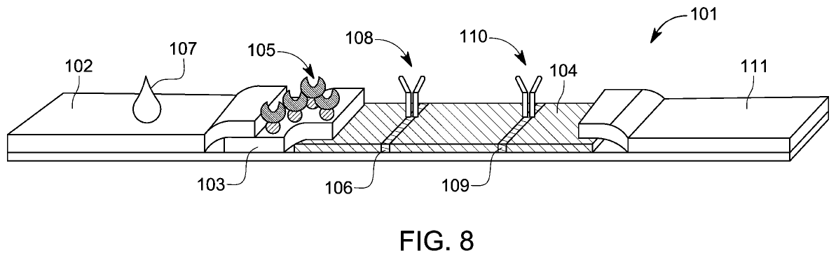

An example of a lateral flow test strip (LFTS) 101 for detecting wild-type APOL1 protein (G0) is shown in FIG. 8. The LFTS includes sample receiving pad 102, conjugate test pad 103, nitrocellulose membrane 104 and wicking pad 111. The sample receiving pad is for receiving a liquid sample, such as, but not limited to, a bodily fluid. The bodily fluid can comprise blood, serum, or plasma. The bodily fluid 107 can be received on the sample receiving pad 102 diluted or undiluted, including at a dilution of 3-20%, as described herein above.

Flow of the sample 107 is mediated by a combination of capillary action through the membranous components from the sample receiving pad 102 to the wicking pad 111, and by pressure from the liquid in the sample receiving pad pushing toward the wicking pad.

The conjugate test pad 103 is in liquid communication with the sample receiving pad 102 and downstream in flow direction from the sample receiving pad. The conjugate test pad 103 comprises a capture reagent 105 deposited thereon. In this example of the lateral flow format, the capture reagent 105 comprises (i) a serum resistance-associated protein (SRA) that selectively binds APOL1 G0 over G1 and G2, and (ii) a detectable reporting group. In another embodiment, the capture reagent 105 comprises (i) an APOL1 specific binding partner, and (ii) a detectable reporting group.

As described above, the SRA protein of the present disclosure is not limited to a particular sequence or recombinant construct, so long as the SRA protein retains the function of binding to APOL1 G0 with a selectivity of at least about 10-fold over binding to G1 and G2.

In some aspects of the lateral flow format assay, the capture reagent 105 deposited on the conjugate test pad 103 can comprise microparticles with the SRA or the APOL1 specific binding partner adsorbed or conjugated thereto. In one embodiment, the microparticles comprise nanocellulose beads. The microparticles can be adsorbed with, for example, mouse anti-HA antibody and then recombinant SRA-Fc-HA.

The way the SRA is presented on the microparticles to enable selective binding to G0 is unlimited, as many methods are known to those of skill in the art. For example, the SRA may be expressed as a recombinant fusion protein with one or more proteins for which antisera or monoclonal antibodies are available. In other examples, presentation of the SRA includes the use of haptens or biotin-streptavidin or by direct conjugation of recombinant protein or binding through poly histidine tags or maltose binding protein fusions or glutathione-S-transferase. In the embodiments where the APOL1 specific binding partner is adsorbed or conjugated to the microparticles, the way that the APOL1 specific binding partner is presented on the microparticles to enable specific binding is similarly unlimited, as many methods are known to those of skill in the art.

In the lateral flow format assays of the present disclosure, the detectable reporting group can generate a measurable signal, such as a radioactive, chromogenic, chemiluminescent, or fluorescent signal. In one aspect, a measurable signal may be produced with an enzyme (e.g., as commonly used in an ELISA). The detectable reporting group can be a visible dye or colored compound or a fluorescent dye. In one aspect, the detectable reporting group is selected from colloidal gold microparticles, latex microparticles, paramagnetic microparticles, and quantum dots.

The nitrocellulose membrane 104 is in liquid communication with the conjugate pad 103 and downstream in flow direction from the conjugate pad 103. The nitrocellulose membrane comprises at a first position 106 a first detection reagent immobilized thereon 108, wherein the first detection reagent 108 comprises an APOL1 specific binding partner. The APOL1 specific binding partner-first detection reagent binds the APOL1 G0 protein while it is bound to the capture reagent 105. In one aspect, the first detection reagent 108 can comprise an anti-ApoL1 antibody (or fragment or derivative thereof) or a peptide or bacteriophage that binds to ApoL1, or an antibody (or fragment or derivative thereof), peptide or bacteriophage that binds to a protein associated with ApoL1.

In another embodiment of the lateral flow format assay where the capture reagent 105 comprises an APOL1 specific binding partner immobilized on the nitrocellulose membrane, the first detection reagent 108 comprises the SRA of the present disclosure that selectively binds APOL1 G0 over G1 and G2.

The nitrocellulose membrane 104 can comprise a second detection reagent 110 immobilized thereon at a second position 109 downstream from the first detection reagent. The second detection reagent 110 is an antibody or antibody fragment or derivative that binds the capture reagent irrespective of whether the capture reagent is bound to APOL1 G0. The second detection reagent can, in some instances, comprise anti-mouse IgG antibody, anti-SRA antibody, or anti-APOL1 specific binding partner antibody, or a fragment or derivative thereof, or a phage or peptide that binds to any portion of the capture reagent 105.

In one aspect of the present disclosure, a lateral flow test strip is provided that includes (a) a sample receiving pad for receiving a liquid sample; (b) a conjugate test pad in liquid communication with the sample receiving pad and downstream in flow direction from the sample receiving pad, wherein the conjugate test pad comprises a capture reagent deposited thereon, and wherein the capture reagent comprises (1) a serum resistance-associated protein (SRA) that selectively binds APOL1 G0 over G1 and G2, and (2) a detectable reporting group; and (c) a nitrocellulose membrane in liquid communication with the conjugate pad and downstream in flow direction from the conjugate pad, wherein the nitrocellulose membrane comprises at a first position a first detection reagent immobilized thereon, wherein the first detection reagent comprises an APOL1 specific binding partner.

In another aspect of the present disclosure, a lateral flow test strip is provided that includes (a) a sample receiving pad for receiving a liquid sample; (b) a conjugate test pad in liquid communication with the sample receiving pad and downstream in flow direction from the sample receiving pad, wherein the conjugate test pad comprises a capture reagent deposited thereon, and wherein the capture reagent comprises (i) an APOL1 specific binding partner, and (ii) a detectable reporting group; and (c) a nitrocellulose membrane in liquid communication with the conjugate pad and downstream in flow direction from the conjugate pad, wherein the nitrocellulose membrane comprises at a first position a first detection reagent immobilized thereon, wherein the first detection reagent comprises a serum resistance-associated protein (SRA) that selectively binds APOL1 G0 over G1 and G2.

In one embodiment, a system is provided for detecting wild-type APOL1 protein (G0), that includes any of the lateral flow test strips described herein above and a test reader to quantitatively determine if the amount of G0 present at the first position is above a predetermined threshold. In one aspect, the predetermined threshold is about 7 nM in the liquid sample.

In another embodiment, a method is provided for assessing APOL1 status in a subject. The method includes depositing a liquid sample from a subject onto the sample receiving pad of any of the lateral flow test strips described herein above, and determining that the subject has at least one wild-type APOL1 G0 allele when the detectable reporting group is visible at the first position, resulting in a positive test result.

In any of the methods of the present disclosure, the subject can be a potential kidney donor. In one aspect, the subject is a potential deceased kidney donor.

In any of the assays, the lateral flow test strips, the systems, and the methods described herein above, the sample can be a liquid sample from a subject. The liquid sample can comprise a bodily fluid from the subject. The bodily fluid can be, for example but not limited to, a blood, serum, or plasma sample, or combinations thereof. The bodily fluid can be diluted or undiluted, including at a dilution of 3-20%.

The following examples serve to illustrate certain aspects of the disclosure and are not intended to limit the disclosure. The contents of all references, pending patent applications and published patents, cited throughout this application are hereby expressly incorporated by reference.

### EXAMPLES

### Example 1: Expression of an SRA Protein Capture Agent and APOL1 G0, G1, and G2

An expression construct for the T. brucei rhodesiense SRA protein (UniProt Q26807) was designed using the nucleotide sequence (Genebank Z37159.2) encoding amino acids 24-410 of SRA. The putative N-terminal secretion sequence was replaced with the N-terminal secretion sequence of PI-16 (UniProt Q6UXB8) and a C-terminal 3× FLAG sequence or Fc-HA sequence was added to the C-terminus. The combined coding regions were synthesized as a single dsDNA fragment (IDT) and cloned into pcDNA3.1 by Gibson cloning. The sequence of the final construct (FIG. 2) was verified by Sanger dideoxy sequencing (GENEWIZ, Morrisville, NC). SRA protein was expressed by transient transfection of HEK 293 cells (ATCC). Briefly, cells cultured in DMEM GlutaMax+10% FBS were transfected using Lipofectamine 3000 (14 μg plasmid DNA per 10-cm dish) and cells were incubated with DNA complexes for 72 h at 37° C. SRA-FLAG and SRA-Fc-HA are predominantly secreted, therefore for use in the assay, supernatant was simply removed from cells and clarified by centrifugation. To improve yields of SRA protein, stably transfected HEK 293 cell lines were established by electroporation of an expression vector, selection was performed with G418 (geneticin), and FACS sorted for high protein expression. Sorted cells were seeded at high density in a CELLine AD 1000 bioreactor and harvested every 3 days. FIG. 2) is a schematic of the recombinant proteins expressed for this study.

Full length APOL1 was constructed similarly to SRA with appropriate amino acid changes or deletions to create the G1 and G2 variants (FIG. 2).^{[4]} APOL1 variants were expressed in a number of cell lines (HEK 293, CHO, HeLa and T47) but levels of G1 and G2 expression were lower than G0 as these proteins were toxic and reduced cell growth. Unlike SRA, APOL1 was not secreted by any of the cell lines tested. Therefore, crude lysates were used as the source of APOL1. Lysates were prepared by scraping cells from a 10 cm dish into 1.5 ml of lysis buffer (PBST (5.6 mM Na₂PO₄, 1 mM KH₂PO₄ pH7.4, 153 mM NaCl, 0.1% Tween 20) supplemented with 0.5% NP40, 1 mM DTT and a protease inhibitor cocktail (Roche), then rotating for 1 h at 4° C. Lysates were clarified by centrifugation before use.

### Example 2: Binding Specificity of SRA for APOL1 G0

To demonstrate the specificity of the SRA capture protein, APOL1 pull-down experiments were performed with immobilized SRA-Fc-HA similar to an immunoprecipitation. Lysates prepared from cells expressing APOL1 variants were normalized with respect to APOL1 concentration and incubated with Protein G Sepharose previously bound with or without SRA-Fc-HA. SRA bound APOL1 G0 but not the G1 or G2 protein variants (FIG. 3), conferring the required specificity for the assay. FIG. 3 is a Western blot of recombinant APOL1 variants incubated with immobilized SRA-Fc-HA on Protein G beads. The membrane was probed with anti-APOL1 antibody (Abcam). Open triangle indicates the correct molecular weight of full length APOL1. Molecular weight markers in kilodaltons.

### Example 3: Assay Specificity and Sensitivity

To assess the sensitivity of the assay for APOL1 G0, lysates were prepared from HEK 293 cells expressing APOL1 G0-HA. The amount of G0 in the lysate was determined by Western blotting and comparison with a purified HA-tagged protein of similar molecular weight. Increasing amounts of lysate resulted in increased signal following detection by the anti-APOL1 antibody (FIG. 4A). FIG. 4A shows a dose response curve for recombinant APOL1 G0 binding to SRA. An ELISA type assay of SRA-FLAG immobilized to anti-FLAG antibody was incubated with increasing amounts of recombinant APOL1 G0 protein produced in a cell lysate. Binding of APOL1 G0 was detected with anti-APOL1 antibody, anti-rabbit HRP and TMB colorimetric substrate (5 min at room temperature). Error bars+/−standard deviation.

FIG. 4B is a graph showing the SRA-based ELISA is specific for the G0 variant of APOL1. ELISA assay using immobilized SRA-FLAG with increasing and equivalent amounts of G0, G1, and G2 isotype extracts normalized by comparing Western blot intensities (see also FIG. 5). A comparison of the dose-response with normalized amounts of APOL1 G0, G1, and G2 shows little response from the G1 and G2 lysates, again demonstrating the specificity of SRA for the G0 variant (FIG. 4B). Control FLAG-tagged proteins produced by the same transfection protocol (CD271 and VAMP8) do not bind APOL1 indicating that SRA protein is responsible for assay specificity (data not shown).

A detection method was developed as a sandwich ELISA with anti-FLAG antibody adsorbed to a microtiter plate to present SRA-FLAG capture reagent. An anti-APOL1 antibody (rabbit monoclonal, Abcam) in combination with HRP conjugated anti-rabbit IgG was used to detect APOL1 bound to the SRA. Plasma diluted to 5% was incubated with immobilized SRA-FLAG. APOL1 binding was detected by anti-APOL1 and anti-rabbit HRP and TMB colorimetric substrate for 5 minutes at room temperature. The results are shown in FIG. 5. ‘Cutoff’ indicates the LoB of the assay used to distinguish a positive or negative result (0.3). The LoB below that represents the reagent LoB (0.12). The results show that the SRA-based ELISA assay correctly identified the presence or absence of APOL1 G0 in 98% of patient samples tested.

Importantly, APOL1 G0 concentrations within patient samples range from 100-1800 nM.^{[25]} The assay has a signal greater than 3-fold above noise at approximately 7 nM, indicating that the assay performs well below the range required for accurate detection of APOL1 G0 the clinic. The LoB (qualitative cutoff) of the assay is demonstrated most clearly in clinical samples, shown in FIG. 5.

Titrated plasma shows a positive response at 3-20% dilution with a maximum response around 5% dilution. Ammonium sulfate fractionation of plasma suggests the presence of an inhibitor is the cause for reduction in signal above 20%. The sensitivity of the assay circumvents this problem and sample dilution further decreases the risk of matrix interference. The assay is also fully functional in 10% whole blood (data not shown).

To validate assay performance in clinically obtained plasma samples from patients, plasma samples of all potential APOL1 risk allele genotype combinations (i.e., G0/G0, G0/G1, G0/G2, G1/G2, G2/G2, and G1/G1) were tested.

Plasma from N=50 African Americans representing all potential genotypes bearing 1-2 risk alleles: APOL1 G0/G1 and G0/G2 (non-risk states) and APOL1 G1/G1, G1/G2 and G2/G2 (risk states) had been previously collected and genotyped. The assay was tested using a 5% dilution of each plasma sample in PBST (N=10 per genotype). Each of the five genotypes is clearly defined by the scale of response in the assay (see FIG. 5). The concordance of the assay results with APOL1 genotype by sequencing is 98% with 49/50 individuals correctly identified as non-risk (signal greater than 0.3, positive for G0) or risk (signal less than 0.3, negative for G0), and no false positives. The cutoff value of 0.3 was determined by a LoB determination derived from 6 pools of G1/G2 plasma that were each measured 10 times. LoB was calculated as the mean of N=60 measurements plus 1.645 times the standard deviation. The LoB (0.12) for reagents alone was similarly determined with PBST in place of plasma (FIG. 5). The assay did provide one false negative result in a G0/G2 plasma genotyped prior to transfer to our lab. It is not possible to confirm if this is a true false negative, as the remaining plasma from this patient contained insufficient DNA for PCR to confirm genotype.

In a separate experiment, plasma was utilized from N=130 African Americans representing all potential genotypes bearing 0-2 risk alleles: APOL1 G0/G0, G0/G1, G0/G2 (non-risk states) and APOL1 G1/G1, G1/G2, G2/G2 (risk states). These plasma samples had been previously collected and genotyped. The assay was tested using a 10% dilution of each plasma sample in sodium acetate buffer. The results show that each of the six genotypes is clearly defined by the scale of response in the assay (see FIG. 6). The concordance of the assay results illustrated in FIG. 6 with APOL1 genotype determined by sequencing is 100% with 90/90 individuals correctly identified as non-risk (signal greater than 1.5, positive for G0) and 40/40 identified as risk (signal less than 1.5, negative for G0).

### Example 4: Assay Conversion to Lateral Flow Format

To demonstrate feasibility of conversion of the ELISA assay to a lateral flow format, nitrocellulose membranes were prepared with anti-ApoL1 and anti-mouse IgG (which binds to anti-FLAG antibody) (FIG. 7). Plasma samples from all six genotypes were diluted to 10% with PBST and mixed with nanocellulose beads adsorbed with first mouse anti-HA antibody and then recombinant SRA-Fc-HA (FIG. 2). Nitrocellulose strips with wicks were placed upright in the bead/plasma suspension and beads allowed to migrate up the strip. Beads were retained by the ApoL1 test spot only if the plasma was G0. Beads mixed with G1 or G2 genotype plasma did not bind anti-ApoL1, since SRA binds more weakly to these variants, but did bind to the positive control line showing that the SRA-beads had migrated past the test spot. The results in FIG. 7 show that the lateral flow format retains selectivity of SRA for the G0 isoform of ApoL1. Duplicate samples of each plasma were mixed with SRA-Fc-HA beads and allowed to migrate across the test area. SRA bead flow is from bottom to top.

While the above disclosure has been described with reference to exemplary embodiments, those of ordinary skill in the art will understand that various changes in form and details may be made without departing from the spirit and scope of the present invention as defined by the following claims.

### REFERENCES

1. Nally, J. V., Jr., Chronic kidney disease in African Americans: Puzzle pieces are falling into place. Cleve Clin J Med, 2017. 84(11): p. 855-862.
2. United States Renal Data System, USRDS annual data report: Epidemiology of kidney disease in the United States, in National Institute of Diabetes and Digestive and Kidney Diseases. 2017, National Institutes of Health: Bethesda, MD.
3. Julian, B. A., et al., Effect of Replacing Race With Apolipoprotein L1 Genotype in Calculation of Kidney Donor Risk Index. Am J Transplant, 2017. 17(6): p. 1540-1548.
4. Genovese, G., et al., Association of trypanolytic ApoL1 variants with kidney disease in African Americans. Science, 2010. 329(5993): p. 841-5.
5. Friedman, D. J., A Brief History of APOL1: A Gene Evolving. Semin Nephrol, 2017. 37(6): p. 508-513.
6. Lan, X., et al., Protein domains of APOL1 and its risk variants. Exp Mol Pathol, 2015. 99(1): p. 139-44.
7. Thomson, R., et al., Evolution of the primate trypanolytic factor APOL1. Proc Natl Acad Sci USA, 2014. 111(20): p. E2130-9.
8. Shah, P. B., et al., APOL1 Polymorphisms in a Deceased Donor and Early Presentation of Collapsing Glomerulopathy and Focal Segmental Glomerulosclerosis in Two Recipients. Am J Transplant, 2016. 16(6): p. 1923-1927.
9. Freedman, B. I., et al., The apolipoprotein L1 (APOL1) gene and nondiabetic nephropathy in African Americans. J Am Soc Nephrol, 2010. 21(9): p. 1422-6.
10. Genovese, G., et al., A risk allele for focal segmental glomerulosclerosis in African Americans is located within a region containing APOL1 and MYH9. Kidney Int, 2010. 78(7): p. 698-704.
11. Nicholas Cossey, L., C. P. Larsen, and H. Liapis, Collapsing glomerulopathy: a 30-year perspective and single, large center experience. Clin Kidney J, 2017. 10(4): p. 443-449.
12. Freedman, B. I. and K. Skorecki, Gene-gene and gene-environment interactions in apolipoprotein L1 gene-associated nephropathy. Clin J Am Soc Nephrol, 2014. 9(11): p. 2006-13.
13. Foster, M. C., et al., APOL1 variants associate with increased risk of CKD among African Americans. J Am Soc Nephrol, 2013. 24(9): p. 1484-91.
14. Freedman, B. I. and B. A. Julian, Should kidney donors be genotyped for APOL1 risk alleles? Kidney Int, 2015. 87(4): p. 671-3.
15. Reeves-Daniel, A. M., et al., The APOL1 gene and allograft survival after kidney transplantation. Am J Transplant, 2011. 11(5): p. 1025-30.
16. Freedman, B. I., et al., APOL1 Genotype and Kidney Transplantation Outcomes From Deceased African American Donors. Transplantation, 2016. 100(1): p. 194-202.
17. O'Toole, J. F., et al., The Cell Biology of APOL1. Semin Nephrol, 2017. 37(6): p. 538-545.
18. Freedman, B. I., et al., Apolipoprotein L1 gene variants in deceased organ donors are associated with renal allograft failure. Am J Transplant, 2015. 15(6): p. 1615-22.
19. Doshi, M. D., et al., APOL1 Genotype and Renal Function of Black Living Donors. J Am Soc Nephrol, 2018. 29(4): p. 1309-1316.
20. Hunter, R. Guidance on Effective Practices in Broader Distribution. 2019 Dec. 26, 2019]; Available from: https://optn.transplant.hrsa.gov/media/2806/osc_publiccomment_20190122.pdf.
21. Gordon, E. J., et al., A National Survey of Transplant Surgeons and Nephrologists on Implementing Apolipoprotein L1 (APOL1) Genetic Testing Into Clinical Practice. Prog Transplant, 2018: p. 1526924818817048.
22. Knight Diagnostic Laboratories. APOL1, Sequencing. 2016 [cited 2017 Dec. 15, 2017]; Available from: https://www.ohsu.edu/custom/knight-diagnostic-labs/home/test-details?id=APOL1%2C+Sequencing.
23. Lindsey, H. The APO-L1 Testing Debate. 2019 Dec. 5, 2019]; Available from: https://www.aacc.org/publications/cln/articles/2019/september/the-apo-11-testing-debate.
24. Madhavan, S. M., et al., APOL1 variants change C-terminal conformational dynamics and binding to SNARE protein VAMP8. JCI Insight, 2017. 2(14).
25. Kozlitina, J., et al., Plasma Levels of Risk-Variant APOL1 Do Not Associate with Renal Disease in a Population-Based Cohort. J Am Soc Nephrol, 2016. 27(10): p. 3204-3219.

1. A method for detecting wild-type ApoL1 protein (G0), comprising:
(a) incubating a sample from a subject being assessed for an ApoL1 G0 protein with:
i) a first binding partner comprising a serum resistance-associated (SRA) protein that selectively binds APOL1 G0 over G1 and G2, and
ii) a second binding partner comprising an ApoL1 specific binding partner, wherein one of either the first or the second binding partner is immobilized on a solid phase, wherein the first or the second binding partner that is not immobilized on the solid phase comprises a detectable label, and wherein the ApoL1 G0 and first and second binding partners form a solid phase binding complex; and
(b) separating the solid phase binding complex from the unbound first or second binding partner comprising the detectable label,
wherein the detectable label associated with the separated complex indicates the presence of ApoL1 G0 in the sample.

2. A system for detecting wild-type ApoL1 protein (G0), comprising:
a. an incubation vessel;
b. a reagent dispensing module; and
c. software to execute the method of claim 1,
wherein the method is executed robotically.

3. The method of claim 1, wherein the sample comprises a bodily fluid, blood, plasma, or serum, and combinations thereof.

4. The method of claim 3, wherein the bodily fluid is at a dilution of 3-20%.

5. The method of claim 1, wherein the subject is a potential kidney donor.

6. The method of claim 1, wherein the solid phase comprises microparticles, nanocellulose beads, or a surface of the incubating well or chamber.

7. The method of claim 6, wherein the microparticles are adsorbed with mouse anti-HA antibody and then recombinant SRA-Fc-HA.

8. The method of claim 1, wherein the second ApoL1 specific binding partner comprises an anti-ApoL1 antibody or a fragment or derivative thereof, an anti-mouse IgG antibody, a phage, or a peptide.

9. The method of claim 1, wherein the detectable label comprises an enzyme, oligonucleotide, nanoparticle, visible dye or colored compound, chemiluminophore, fluorophore, fluorescence quencher, chemiluminescence quencher, or biotin, and combinations thereof.

10. (canceled)

11. (canceled)

12. A lateral flow test strip for detecting wild-type APOL1 protein (G0), comprising:
(a) a sample receiving pad for receiving a liquid sample;
(b) a conjugate test pad in liquid communication with the sample receiving pad and downstream in flow direction from the sample receiving pad, wherein the conjugate test pad comprises a capture reagent deposited thereon, and wherein the capture reagent comprises (i) a serum resistance-associated protein (SRA) that selectively binds APOL1 G0 over G1 and G2, and (ii) a detectable reporting group; and
(c) a nitrocellulose membrane in liquid communication with the conjugate pad and downstream in flow direction from the conjugate pad, wherein the nitrocellulose membrane comprises at a first position a first detection reagent immobilized thereon, wherein the first detection reagent comprises an APOL1 specific binding partner.

13. A lateral flow test strip for detecting wild-type APOL1 protein (G0), comprising:
(a) a sample receiving pad for receiving a liquid sample;
(b) a conjugate test pad in liquid communication with the sample receiving pad and downstream in flow direction from the sample receiving pad, wherein the conjugate test pad comprises a capture reagent deposited thereon, and wherein the capture reagent comprises (i) an APOL1 specific binding partner, and (ii) a detectable reporting group; and
(c) a nitrocellulose membrane in liquid communication with the conjugate pad and downstream in flow direction from the conjugate pad, wherein the nitrocellulose membrane comprises at a first position a first detection reagent immobilized thereon, wherein the first detection reagent comprises a serum resistance-associated protein (SRA) that selectively binds APOL1 G0 over G1 and G2.

14. A system for detecting wild-type APOL1 protein (G0), comprising:
(a) the lateral flow test strip of claim 12; and
(b) a test reader to quantitatively determine if the amount of G0 present at the first position is above a predetermined threshold.

15. The lateral flow test strip of claim 12, wherein the nitrocellulose membrane further comprises a second detection reagent immobilized thereon at a second position downstream from the first detection reagent, wherein the second detection reagent is an antibody or a fragment or derivative thereof, a phage or a peptide that binds the capture reagent irrespective of whether the capture reagent is bound to APOL1 G0.

16. The lateral flow test strip of claim 15, wherein the second detection reagent comprises anti-mouse IgG antibody.

17. The lateral flow test strip of claim 12, wherein the APOL1 specific binding partner comprises an anti-ApoL1 antibody or a fragment or derivative thereof, an anti-mouse IgG antibody, a phage, or a peptide.

18. The lateral flow test strip of claim 12, wherein the capture reagent deposited on the conjugate test pad comprises microparticles with the SRA or APOL1 specific binding partner adsorbed or conjugated thereto.

19. The lateral flow test strip of claim 18, wherein the microparticles are adsorbed with mouse anti-HA antibody and then recombinant SRA-Fc-HA.

20. (canceled)

21. (canceled)

22. A method of assessing APOL1 status in a subject, the method comprising:
a. depositing a liquid sample from a subject onto the sample loading pad of the lateral flow test strip of claim 12; and
b. determining that the subject has at least one wild-type APOL1 G0 allele when the detectable reporting group is visible at the first position, resulting in a positive test result.

23. The method of claim 22, wherein the subject is a potential kidney donor.

24. The method of claim 22, wherein the subject is a potential deceased kidney donor.

25. (canceled)

26. The method of claim 22, wherein the liquid sample comprises a blood, serum, or plasma sample.
